(19) United States
(12) Patent Application Publication
(10) Pub. No.: US 2023/0381498 A1
(43) Pub. Date: Nov. 30, 2023

(54) THREE-DIMENSIONAL RETINAL STIMULATION DEVICE

(71) Applicants: KOREA UNIVERSITY RESEARCH AND BUSINESS FOUNDATION, Seoul (KR); DAEGU GYEONGBUK INSTITUTE OF SCIENCE AND TECHNOLOGY, Daegu (KR)

(72) Inventors: Seong Woo KIM, Seoul (KR); So Hee KIM, Daegu (KR); Hee Won SEO, Gyeonggi-do (KR)

(21) Appl. No.: 18/031,314

(22) PCT Filed: Sep. 1, 2021
(86) PCT No.: PCT/KR2021/011718

§ 371 Date: Apr. 11, 2023

(30) Foreign Application Priority Data
Oct. 12, 2020 (KR) 10-2020-0130910

(51) Int. Cl. A61N 1/05

(52) U.S. Cl. CPC A61N 1/0543

(57) ABSTRACT

A three-dimensional retinal stimulation device includes a substrate having a top surface, a bottom surface, and a plurality of edges connecting the top surface and the bottom surface to each other; and a plurality of electrodes disposed between the top surface and the bottom surface, wherein the plurality of edges include inclined surfaces with respect to the top surface and the bottom surface.

### TECHNICAL FIELD

The following embodiments relate to a three-dimensional retinal stimulation device.

### BACKGROUND ART

A sub-retinal artificial stimulation device that is inserted into the eyeball is provided as a technology for improving vision of patients who lose vision due to death of retinal photoreceptor cells. This device converts an optical signal input to the eye into an electrical signal to finally stimulate living retinal ganglion cells in the retina and transmits an image to the brain. For example, U.S. Laid-open Patent Publication No. 2018/0326214 discloses a device for stimulating optic nerve fibers.

### DISCLOSURE OF THE INVENTION

Technical Goals

An object according to an embodiment is to provide a three-dimensional retinal stimulation device for minimizing a mechanical pressure applied to the retina by an external structure of the device inserted into a sub-retinal space.

### Technical Solutions

A three-dimensional (3D) retinal stimulation device according to an embodiment includes a substrate having a top surface, a bottom surface, and a plurality of edges connecting the top surface and the bottom surface, and a plurality of electrodes provided between the top surface and the bottom surface. The plurality of edges includes an inclined surface with respect to the top surface and the bottom surface.

An angle between the top surface and the inclined surface may be 120 degrees or more and an angle between the bottom surface and the inclined surface may be 60 degrees or less.

The plurality of edges may include a vertical portion between the inclined surface and the bottom surface.

The inclined surface may be formed of a flat portion that does not include a protruding portion.

The top surface may include a plurality of curved portions formed concavely from a side surface of an upper portion of the plurality of electrodes toward the bottom surface.

A length of a first edge of the plurality of edges may be different from a length of a second edge adjacent to the first edge.

A height of the plurality of electrodes may be 40 μm or less.

A method of manufacturing the 3D retinal stimulation device according to an embodiment includes depositing a metal for forming a plurality of electrodes on a substrate and forming a plurality of holes between the plurality of electrodes by patterning the substrate, filling the plurality of holes with a filling material, arranging an elastic lid on the substrate, and pressing the lid against the substrate and adjusting a height between a low point of the filling material and the plurality of electrodes formed by the lid by adjusting pressure of the lid against the substrate.

The height between the low point of the filling material and the plurality of electrodes may be adjusted by adjusting the pressure of the lid by a binder clip when pressing the lid against the substrate.

Effects

The 3D retinal stimulation device according to an embodiment may minimize mechanical pressure applied to the retina due to an external structure of the device input to a sub-retinal space, thereby minimizing damage to cell layers on the device.

The 3D retinal stimulation device according to an embodiment may minimize damage to the retinal tissues caused by the 3D retinal stimulation device while maximally increasing a charge density of electrodes positioned in the sub-retinal space.

The effects of the 3D retinal stimulation device according to an embodiment are not limited to the above-mentioned effects, and other unmentioned effects can be clearly understood from the above description by those having ordinary skill in the technical field to which the present disclosure pertains.

### BRIEF DESCRIPTION OF DRAWINGS

FIG. 1 is a diagram illustrating an example of using a three-dimensional (3D) retinal stimulation device according to an embodiment.

FIG. 2 is a perspective view of a 3D retinal stimulation device according to an embodiment.

FIG. 3 is a cross-sectional view of the 3D retinal stimulation device of FIG. 2 taken along line A-A.

FIG. 4 is an enlarged cross-sectional view of a section B of the 3D retinal stimulation device of FIG. 3.

FIG. 5 is a plan view of the 3D retinal stimulation device of FIG. 2.

FIGS. 6A to 6H are diagrams illustrating a method of manufacturing a 3D retinal stimulation device according to an embodiment.

FIGS. 7 and 8 are diagrams illustrating a specific method of step (c) of FIGS. 6A to 6H.

FIGS. 9A to 9E are diagrams illustrating a specific method of steps (f) and (g) of FIGS. 6A to 6H.

FIGS. 10A to 10C are diagrams illustrating a specific method of step (h) of FIGS. 6A to 6H.

FIG. 11 is a photograph of a top surface of a 3D retinal stimulation device manufactured by the method of FIGS. 6A to 6H.

### BEST MODE FOR CARRYING OUT THE INVENTION

Hereinafter, embodiments will be described in detail with reference to the accompanying drawings. However, various alterations and modifications may be made to the embodiments. Here, the embodiments are not construed as limited to the disclosure. The embodiments should be understood to include all changes, equivalents, and replacements within the idea and the technical scope of the disclosure.

The terminology used herein is for the purpose of describing particular embodiments only and is not to be limiting of the embodiments. The singular forms “a”, “an”, and “the” are intended to include the plural forms as well, unless the context clearly indicates otherwise. It will be further understood that the terms “comprises/comprising” and/or “includes/including” when used herein, specify the presence of stated features, integers, steps, operations, elements, and/or components, but do not preclude the presence or addition of one or more other features, integers, steps, operations, elements, components and/or groups thereof.

Unless otherwise defined, all terms including technical and scientific terms used herein have the same meaning as commonly understood by one of ordinary skill in the art to which embodiments belong. It will be further understood that terms, such as those defined in commonly-used dictionaries, should be interpreted as having a meaning that is consistent with their meaning in the context of the relevant art and will not be interpreted in an idealized or overly formal sense unless expressly so defined herein.

When describing the embodiments with reference to the accompanying drawings, like reference numerals refer to like constituent elements and a repeated description related thereto will be omitted. In the description of embodiments, detailed description of well-known related structures or functions will be omitted when it is deemed that such description will cause ambiguous interpretation of the present disclosure.

Also, in the description of the components, terms such as first, second, A, B, (a), (b) or the like may be used herein when describing components of the present disclosure. These terms are used only for the purpose of discriminating one constituent element from another constituent element, and the nature, the sequences, or the orders of the constituent elements are not limited by the terms. When one constituent element is described as being “connected”, “coupled”, or “attached” to another constituent element, it should be understood that one constituent element can be connected or attached directly to another constituent element, and an intervening constituent element can also be “connected”, “coupled”, or “attached” to the constituent elements.

The same name may be used to describe an element included in the embodiments described above and an element having a common function. Unless otherwise mentioned, the descriptions of the embodiments may be applicable to the following embodiments and thus, duplicated descriptions will be omitted for conciseness.

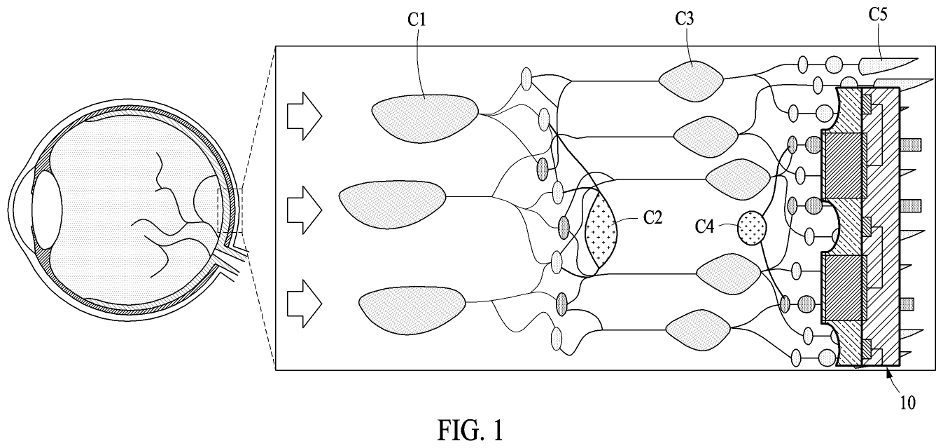

Referring to FIG. 1, a three-dimensional (3D) retinal stimulation device 10 according to an embodiment may be inserted into the eyeball of a subject to transfer an electrical signal to the retina. Here, the subject may include a living organism such as a human or an animal. For example, the 3D retinal stimulation device 10 may be inserted into a sub-retinal space including ganglion cells C1, amacrine cells C2, bipolar cells C3, horizontal cells C4, and photoreceptor cells C5 of the eyeball of the subject. For example, if the photoreceptor cells C5 are damaged, the 3D retinal stimulation device 10 may be inserted into a space between the bipolar cells C3 and the photoreceptor cells C5.

A normal retinal thickness is known to be approximately 250 micrometers (μm). A thickness of the diseased retina, into which the 3D retinal stimulation device 10 is inserted, is generally less than 150 μm. which is extremely smaller than the normal retinal thickness. Therefore, the 3D retinal stimulation device 10 may be manufactured to have an average thickness of 70 μm in consideration of the thickness of the diseased retina. An edge of the 3D retinal stimulation device 10 manufactured to have such a thickness does not have a rectangular cross section, which is a general shape, but has a cross section with a curved surface or an inclined surface. This may prevent compression due to the edge of the 3D retinal stimulation device 10 when the 3D retinal stimulation device 10 is inserted into an extremely thin sub-retinal space and the retina above the 3D retinal stimulation device 10 is mounted on the 3D retinal stimulation device 10. As a result, the 3D retinal stimulation device 10 may prevent retinal folds and compression when the 3D retinal stimulation device 10 is inserted into the eyeball, may maintain nutrition supplies through retinal fiber layers corresponding to axons of the ganglion cells of the retina, and may maintain the function of the 3D retinal stimulation device 10 in the long term by preventing shrinkage of the ganglion cells that are the target of electrical stimulation of the 3D retinal stimulation device 10.

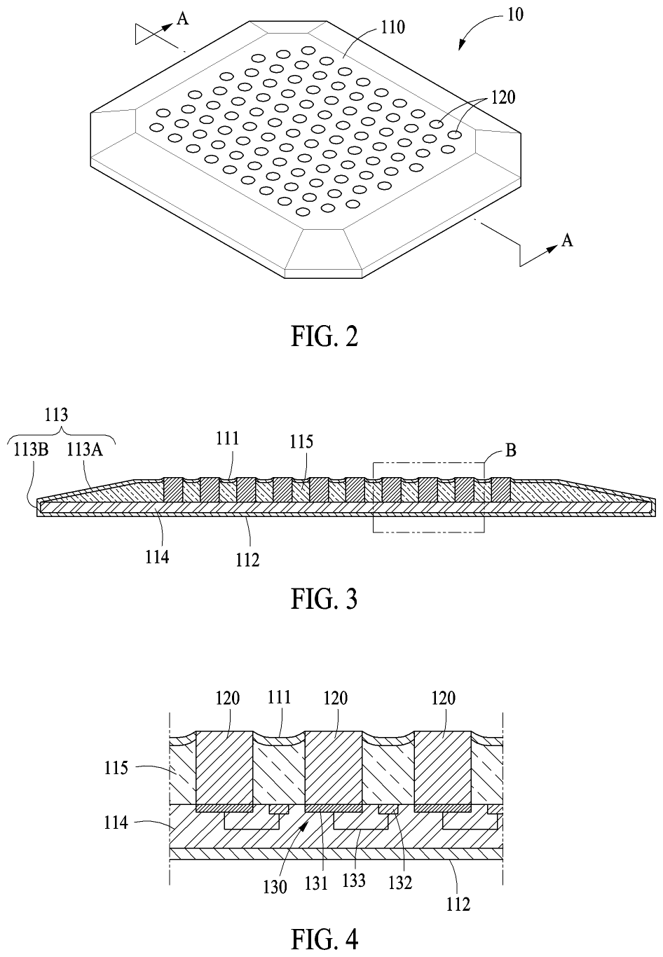

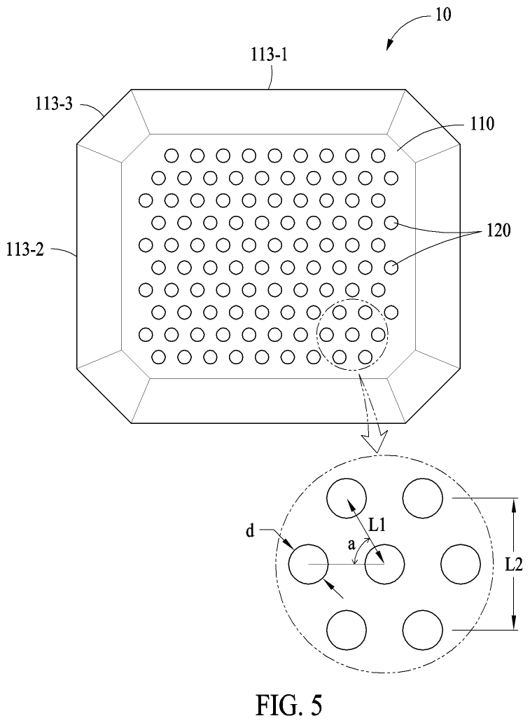

Referring to FIGS. 2 to 5, the 3D retinal stimulation device 10 includes a substrate 110, a plurality of electrodes 120, and a circuit unit 130.

The substrate 110 includes a top surface 111, a bottom surface 112, and a plurality of edges 113 connecting the top surface 111 and the bottom surface 112. The top surface 111, the bottom surface 112, and the plurality of edges 113 may define a cavity of the substrate 110.

The top surface 111 and the bottom surface 112 may be substantially parallel to each other. An area of the top surface 111 may be smaller than an area of the bottom surface 112. For example, a length of a portion where the top surface 111 and the plurality of edges 113 meet is approximately 4 millimeters (mm) or less, and a length of a portion where the bottom surface 112 and the plurality of edge 113 meet is approximately 5 mm or less or approximately 4.5 mm or less. The top surface 111 and the bottom surface 112 are spaced apart from each other. Here, a distance between the top surface 111 and the bottom surface 112 may be significantly smaller than the area of the top surface 111 or the area of the bottom surface 112.

The top surface 111 may include a planar portion and a plurality of curved portions. The plurality of curved portions may be formed between the plurality of electrodes 120, respectively, and each thereof may be formed concavely in a direction from a side surface of an upper portion of each of the electrodes 120 toward the bottom surface 112.

The plurality of edges 113 may have a predetermined shape that is suitable to prevent tissue damage. The plurality of edges 113 may include an inclined surface 113A. The inclined surface 113A may be formed substantially smooth and flat without any protruding portions. The inclined surface 113A may be angled at an angle of 120 degrees or more with respect to the top surface 111 and may be angled at an angle of 60 degrees or less with respect to the bottom surface 112. A horizontal length of the plurality of edges 113 may be approximately 0.5 mm or less. This may enable the retinal tissue to cover the 3D retinal stimulation device 10 without a rapid angle change and without applying physical pressure when the 3D retinal stimulation device 10 is inserted into the retina. This may also make it easy to manufacture the 3D retinal stimulation device 10 thick enough to meet engineering requirements.

The plurality of edges 113 may further include a vertical portion 113B between the inclined surface 113A and the bottom surface 112. The vertical portion 113B may be substantially perpendicular to the bottom surface 112. The geometric shape of the cavity of the substrate 110 formed by the inclined surface 113A and the vertical portion 113B may help support the plurality of electrodes 120 within the cavity of the substrate 110.

The plurality of edges 113 may have a polygonal shape when the 3D retinal stimulation device 10 is viewed from above. For example, the plurality of edges 113 may include a horizontal edge portion 113-1, a vertical edge portion 113-2, and a connection edge portion 113-3 connecting the horizontal edge portion 113-1 and the vertical edge portion 113-2. The horizontal edge portion 113-1 may have a length of approximately 5 mm or less, and the vertical edge portion 113-2 may have a length of approximately 4.5 mm or less. Here, the length of the horizontal edge portion 113-1 may be greater than the length of the vertical edge portion 113-2. The horizontal edge portion 113-1, the vertical edge portion 113-2, and the connection edge portion 113-3 may form the plurality of edges 113 to have a shape that is similar to a shape with round edges as a whole.

The top surface 111, the bottom surface 112, and the plurality of edges 113 may be formed of a predetermined material suitable to be inserted into the retina. For example, the top surface 111, the bottom surface 112, and the plurality of edges 113 may be formed of parylene and may form a coating layer of the substrate 110. The coating layer may have a thickness of approximately 3 μm.

The substrate 110 may include a first material layer 114 and a second material layer 115 disposed inside the cavity. The first material layer 114 and the second material layer 115 may be sequentially stacked in a direction from the bottom surface 112 toward the top surface 111. The first material layer 114 may be disposed on the bottom surface 112 and may be formed as high as the vertical portion 113B of the plurality of edges 113. For example, the first material layer 114 may have a thickness of approximately 40 μm or less and may include a polymer film. The second material layer 115 may be disposed between the first material layer 114 and the top surface 111 and may form a space of the cavity of the substrate 110 that is remaining after being filled with the first material layer 114. For example, the second material layer 115 may be a polydimethylsiloxane (PDMS) layer.

Alternatively, the first material layer 114 may not include a material, in which case the first material layer 114 may also be referred to as a “first layer”. The first layer may be formed as the circuit unit 130 including an optical sensor 132 such as a photodiode that receives light from the outside and converts the light into a current, and a circuit board 131 such as an integrated circuit including a current generator (not shown) that generates a current for the retinal stimulation and amplifies a magnitude thereof. In this case, the first layer may include silicon.

The plurality of electrodes 120 are configured to generate electrical signals. The plurality of electrodes 120 may be accommodated in the cavity of the substrate 110 and arranged in a matrix form over substantially the entire portion of the substrate 110. Each of the electrodes 120 may have a substantially cylindrical shape. The plurality of electrodes 120 may be placed on the first material layer 114, supported by the first material layer 114, and buried in the second material layer 115. In addition, the plurality of electrodes 120 may extend to the top surface 111.

The plurality of electrodes 120 may have a predetermined height suitable for preventing damage to the retinal tissue. For example, the height of the plurality of electrodes 120 between a lower end portion where the plurality of electrodes 120 meets the first material layer 114 and an upper end portion where the plurality of electrodes 120 meets the top surface 111 may be approximately 50 μm or more and approximately 160 μm or less. Such a height of the plurality of electrodes 120 is determined in consideration of the point that a subject with a retinal disease (e.g., retinal degeneration) requiring implants of the 3D retinal stimulation device 10 has the retina having a thickness of approximately less than 120 μm and the point that a thickness of a bipolar cell layer of the retina is 50 μm or more and less than 60 μm, and may thus minimize mechanical pressure applied to the retinal nerve by the plurality of electrodes 120.

A height of the plurality of electrodes 120 from a low point of a curved surface of the top surface 111 to an upper end of the electrode 120 may be approximately 40 μm or less, approximately 30 μm or less, or approximately 20 μm or less. In a preferable example, the height may be approximately 20 μm or less. This may suppress the acceleration of a retinal fibrosis reaction that may occur due to continuous frictional contact between the upper end portion of the plurality of electrodes 120 and the retinal tissue and prevent damage to the retinal nerve, even if the upper end portion of the plurality of electrodes 120 has a predetermined shape.

The plurality of electrodes 120 may have a shape suitable for preventing the damage to the retinal tissue. In an example not shown, the upper end portion of the plurality of electrodes 120 may be formed in a substantially hemispherical shape. This may also minimize the mechanical pressure applied to the retinal nerve by the plurality of electrodes 120.

The plurality of electrodes 120 may be alternately arranged in a plurality of rows in a vertical direction when the 3D retinal stimulation device 10 is viewed from above. In other words, the plurality of electrodes 120 in a first row may not overlap the plurality of electrodes 120 in a second row when viewed in the vertical direction. According to an embodiment, a distance between a pair of adjacent electrodes 120 in one row and a distance L1 between the electrode 120 in one row and the electrode 120 in an adjacent row may be approximately 350 μm, an angle a between a direction along each electrode 120 belonging to one row and a line connecting a pair of adjacent electrodes 120 in one row may be approximately 60°, a distance L2 between two electrodes in two rows adjacent to one row may be approximately 606 μm, and a diameter of each electrode 120 may be approximately 150 μm.

The circuit unit 130 may include the circuit board 131, the optical sensor 132, and an electrical lead 133. The circuit board 131 is disposed under the electrode 120 at an upper interface side of the first material layer 114 and is configured to control the operation of the electrode 120. The optical sensor 132 is configured to detect an optical signal coming from the outside of the 3D retinal stimulation device 10. For example, the optical sensor 132 may include a photodiode. The electrical lead 133 may electrically connect the circuit board 131 and the optical sensor 132 and transmit an optical signal received by the optical sensor 132 to the circuit board 131. Therefore, when the optical sensor 132 receives an optical signal, the received optical signal may be transmitted to the circuit board 131 through the electrical lead 133, and the circuit board 131 may control the electrode 120 so that the electrode 120 emits an electrical stimulation signal.

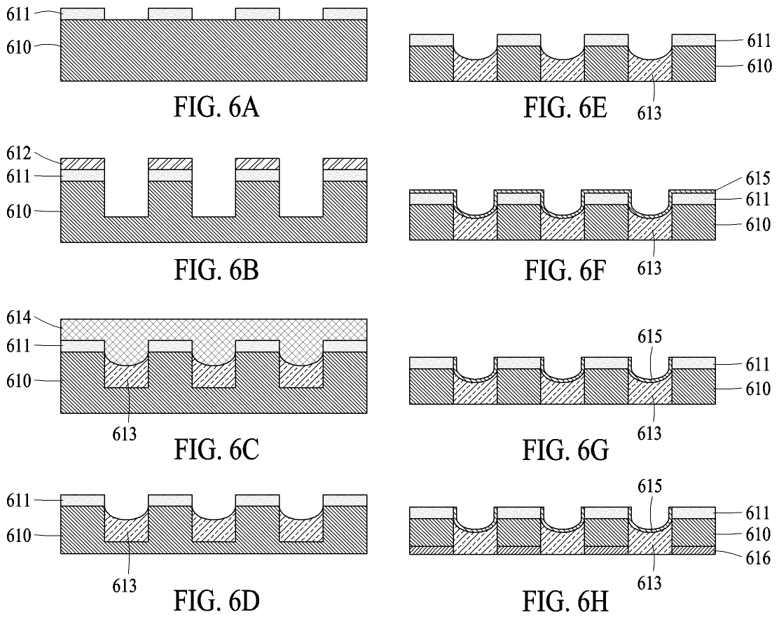

Referring to FIGS. 6A to 6H, in a method of manufacturing a 3D retinal stimulation device, first, in step (a), a material 611 containing or composed of titanium (Ti), platinum (Pt), and iridium oxide (IrOx) is deposited on a silicon wafer 610, and a desired electrode pad pattern is formed using a photoresist patterning process. Here, a shape of the electrode pad pattern may be circle or rectangle.

In step (b), a patterning process of a photoresist 612 for a deep reactive-ion-etching (deep RIE) process is performed on the formed electrode pad pattern. Then, a hole of a transparent substrate is formed by the deep RIE process.

In step (c), the inside of the hole is filled with polydimethylsiloxane (PDMS) 613, which is a transparent material, to manufacture a flexible and transparent substrate. At this time, an elastic lid 614 covers the holes formed by the deep RIE process and the lid is pressed by a pressure of a binder clip, thereby adjusting a height of the polydimethylsiloxane 613 filled therein. The lid 614 may include a plurality of protruding portions having a shape corresponding to the shape of the hole, for example, a convex shape, and a planar portion connecting the plurality of protruding portions.

In step (d), a rear side of the unprocessed silicon wafer 610 is physically etched. For example, silicon present on the rear side may be removed by grinding.

In step (e), the transparent substrate is exposed by performing a chemical etching process on the rear side of the silicon wafer 610. For example, silicon may be removed isotropically using a solution containing hydrofluoric acid and nitric acid.

In step (f), parylene 615, which has biocompatibility and is a material for increasing selective stimulation of each electrode, is coated.

In step (g), a portion of the coated parylene 615 is removed, except for the electrode portion for stimulation. Here, the removal of the parylene 615 may be performed by a RIE process, and a photoresist patterning process may be used to remove the parylene 615.

In step (h), a material 616 containing or composed of titanium (Ti) and gold (Au) is deposited on the back side of the silicon wafer 610 for electrical conductivity. Here, a patterned polymer mask may be used.

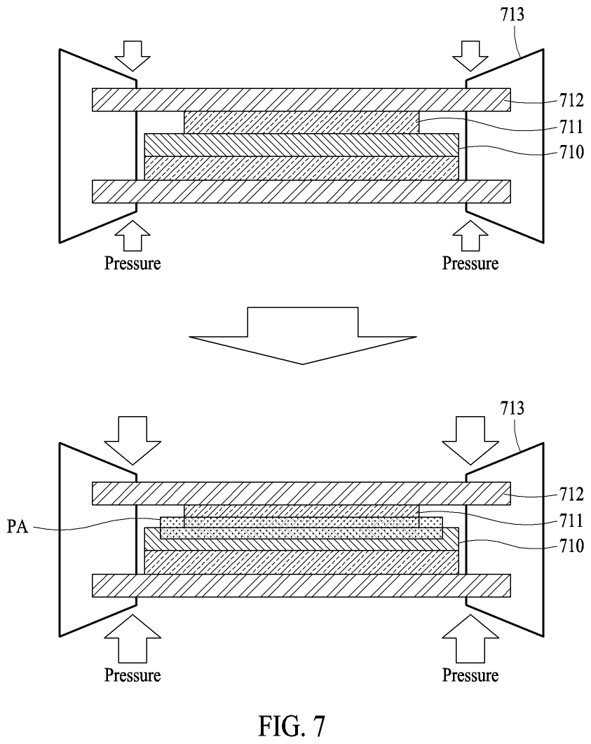

Referring to FIG. 7, in step (c), an elastic lid 711 formed of polydimethylsiloxane (PDMS) is placed on a chip-shaped silicon wafer 710. Then, the elastic lid 711 is pressed into an area PA between the holes by applying the pressure to glass 712 with a binder clip 713. Here, when polydimethylsiloxane (PDMS) is well filled between the holes, the lid 711 may be coated with parylene for easy removal.

When the lid 711 has constant elasticity, the lid 711 is more strongly pressed into the areas between the processed holes, as the pressure of the binder clip 713 increases. As a result, a protrusion height of the electrode may increase.

Meanwhile, when the pressure of the binder clip 713 is constant, a degree of pressing the areas between the holes varies depending on elastic strength of the lid 711, and thus, the shape of the protruding electrode may be adjusted. The elastic strength of lid 711 may depend on a mixing ratio, hardening time, and temperature.

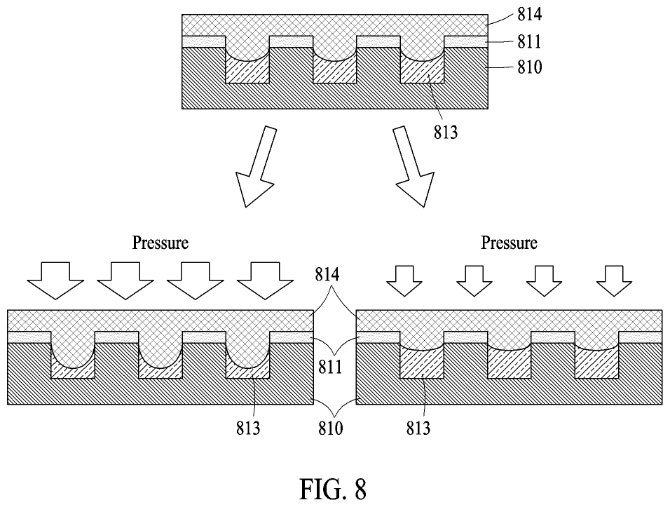

FIG. 8 shows that a plurality of holes formed on a silicon wafer 810 are filled with polydimethylsiloxane (PDMS) 813, and a material 811 containing or composed of titanium (Ti), platinum (Pt), and iridium oxide (IrOx) is provided on an upper surface of the silicon wafer 810. Here, it shows that a height of a 3D electrode formed when the pressure of the binder clip for an elastic lid 814 is strong or the elastic strength of the lid 814 is weak is higher than a height of a 3D electrode formed when the pressure of the binder clip for the lid 814 is weak or the elastic strength of the lid 814 is strong.

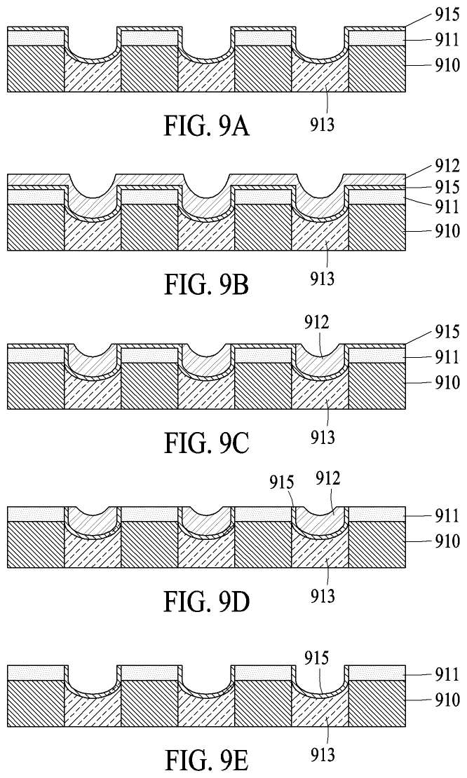

FIGS. 9A to 9E show that a plurality of holes formed on a silicon wafer 910 are filled with polydimethylsiloxane (PDMS) 913, and a material 911 containing or composed of titanium (Ti), platinum (Pt), and iridium oxide (IrOx) is provided on an upper surface of the silicon wafer 910. In step (a), the silicon wafer 910 is coated with parylene 915. Then, in steps (b) and (c), the patterning is performed using a photoresist 912 to expose only the electrode portion. Next, in step (d), the parylene 915 on the electrode portion is removed by the RIE process. Finally, in step (e), the photoresist 912 is removed.

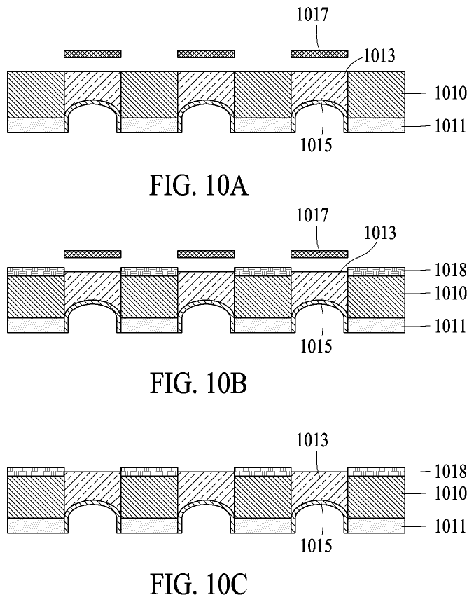

Referring to FIGS. 10A to 10C, as a specific method of step (h) of FIGS. 6A to 6H, a process of depositing a metal on a back side of the electrode may be performed. Here, a reference numeral 1010 denotes a silicon wafer, a reference numeral 1011 denotes a material containing or composed of titanium (Ti), platinum (Pt), and iridium oxide (IrOx), a reference numeral 1013 denotes polydimethylsiloxane, and a reference numeral 1015 denotes parylene. First, in step (a), in order to deposit a metal on a back side of the electrode 1010, a mask 1017 formed of a polymer material is provided to cover. Then, in step (b), the back side of the electrode is exposed by the patterned mask 1017, and a material 1018 containing or composed of titanium (Ti) and gold (Au) is deposited by a sputtering process. Finally, the mask 1017 is removed.

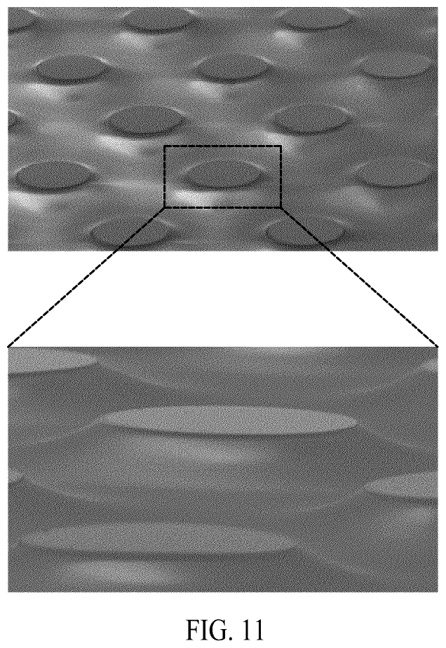

FIG. 11 shows a photograph of a 3D retinal stimulation device manufactured by the method described above with reference to FIGS. 6A to 6H, 7, 8, 9A to 9E and 10A to 10C. A top surface of the device has a shape such as embossing and a height between a protruding portion and a low point of a curved surface may be considered as a substantial height of an electrode. Such a height of the electrode may be adjusted by the process described above.

According to the structural features and methodological features of the device described above, there is an advantage in that a high-resolution electrode may be manufactured with a substrate having a limited size.

On the other hand, when an electrode is inserted between photoreceptor cells in a sub-retinal approach, the photoreceptor cells may be damaged if the protruding electrode is shape, and accordingly, it is necessary to consider that efficient stimulation delivery may not be performed. Therefore, the shape of the protruding electrode to be inserted into the photoreceptor cell is important, and the device of the present embodiment has an advantage of having a 3D electrode with a gentle inclination capable of minimizing the damage to the photoreceptor cells.

In addition, in order to manufacture the 3D electrode with a gentle inclination, the holes are formed by the deep RIE process and the inside of the holes are filled with polydimethylsiloxane, which is a transparent material, using an elastic lid, thereby manufacturing a flexible and transparent substrate. Here, the binder clip may fix the lid to fill the polydimethylsiloxane, and the protrusion height of the electrode may be determined according to the pressure of the binder clip and the elastic strength of the lid.

Meanwhile, since the electrode is inserted between the photoreceptor cells, a thickness of the substrate needs to be thin considering a thickness of the photoreceptor cell. By strongly pressing the areas between the holes using the elastic lid, a space to be filled with polydimethylsiloxane may be reduced, and accordingly, the thickness of the substrate may also be reduced.

In addition, only one type of etching solution is used, unlike the electrode of the related art. Accordingly, the process is relatively simple, and the burden applied to a surface of a wafer during the process is small, and thus, it is advantageous that the chemical damage is reduced.

While the embodiments are described with reference to drawings, it will be apparent to one of ordinary skill in the art that various alterations and modifications in form and details may be made in these embodiments without departing from the spirit and scope of the claims and their equivalents. For example, suitable results may be achieved if the described techniques are performed in a different order, and/or if components in a described system, architecture, device, or circuit are combined in a different manner, and/or replaced or supplemented by other components or their equivalents.

Therefore, other implementations, other embodiments, and/or equivalents of the claims are within the scope of the following claims.

1. A three-dimensional (3D) retinal stimulation device comprising:
a substrate having a top surface, a bottom surface, and a plurality of edges connecting the top surface and the bottom surface; and
a plurality of electrodes provided between the top surface and the bottom surface,
wherein the plurality of edges comprises an inclined surface with respect to the top surface and the bottom surface.

2. The 3D retinal stimulation device of claim 1, wherein an angle between the top surface and the inclined surface is 120 degrees or more and an angle between the bottom surface and the inclined surface is 60 degrees or less.

3. The 3D retinal stimulation device of claim 1, wherein the plurality of edges comprises a vertical portion between the inclined surface and the bottom surface.

4. The 3D retinal stimulation device of claim 1, wherein the inclined surface is formed of a flat portion that does not include a protruding portion.

5. The 3D retinal stimulation device of claim 1, wherein the top surface comprises a plurality of curved portions formed concavely from a side surface of an upper portion of the plurality of electrodes toward the bottom surface.

6. The 3D retinal stimulation device of claim 1, wherein a length of a first edge of the plurality of edges is different from a length of a second edge adjacent to the first edge.

7. The 3D retinal stimulation device of claim 1, wherein a height of the plurality of electrodes is 40 μm or less.

8. A method of manufacturing the 3D retinal stimulation device of claim 1, the method comprising:
depositing a metal for forming a plurality of electrodes on a substrate and forming a plurality of holes between the plurality of electrodes by patterning the substrate;
filling the plurality of holes with a filling material;
arranging an elastic lid on the substrate; and
pressing the lid against the substrate and adjusting a height between a low point of the filling material and the plurality of electrodes formed by the lid by adjusting pressure of the lid against the substrate.

9. The method of claim 8, wherein the height between the low point of the filling material and the plurality of electrodes is adjusted by adjusting the pressure of the lid by a binder clip when pressing the lid against the substrate.
